(19) United States
(12) Patent Application Publication
(10) Pub. No.: US 2014/0018452 A1
(43) Pub. Date: Jan. 16, 2014

(54) CATALYSTS FOR THE CONVERSION OF SYNTHESIS GAS TO ALCOHOLS

(71) Applicants: Dean Millar, Midland, MI (US); Mark H. McAdon, Midland, MI (US); Robert J. Gulotty,, JR., Glendale, AZ (US); David G. Barton, Midland, MI (US); Daniela Ferrari, Antwerp (BE); Billy B. Bardin, Lake Jackson, TX (US); Yu Liu, Lake Jackson, TX (US)

(72) Inventors: Dean Millar, Midland, MI (US); Mark H. McAdon, Midland, MI (US); Robert J. Gulotty,, JR., Glendale, AZ (US); David G. Barton, Midland, MI (US); Daniela Ferrari, Antwerp (BE); Billy B. Bardin, Lake Jackson, TX (US); Yu Liu, Lake Jackson, TX (US)

(73) Assignee: DOW GLOBAL TECHNOLOGIES LLC, Midland, MI (US)

(21) Appl. No.: 14/008,197

(22) PCT Filed: Apr. 1, 2011
(86) PCT No.: PCT/US11/30912

§ 371 Date: Sep. 27, 2013

(51) Int. Cl. C07C 29/48; C07C 45/50; B01J 23/89

(52) U.S. Cl. CPC C07C 29/48; B01J 23/8993; B01J 23/8946; C07C 45/50
USPC 518/713; 502/243; 518/714; 518/717

(57) ABSTRACT

A catalyst suitable for manufacturing a mixture of alcohols from synthesis gas comprises a combination of nickel, two or more metals selected from ruthenium, palladium, gold, chromium, aluminum and tin, and at least one of an alkali metal or alkaline earth series metal as a promoter. The catalyst may be used in a process for converting synthesis gas wherein the primary product is a mixture of ethanol (EtOH), propanol (PrOH), and butanol (BuOH), optionally in conjunction with higher alcohols.

In various aspects, this invention relates generally to a catalyst for converting synthesis gas (or “syngas”, a mixture of gases consisting mainly of carbon monoxide (CO) and hydrogen (H₂)) into a mixture of alcohols (e.g., ethanol (EtOH), propanol (PrOH), and butanol (BuOH), optionally in conjunction with higher alcohols). In various aspects, this invention relates particularly to such a catalyst comprising a combination of nickel, and at least (≧) two or more metals selected from a group consisting of ruthenium, palladium, gold, chromium, aluminum and tin. The catalyst is preferably promoted with an alkali or alkaline earth series metal.

Ethanol and mixtures of alcohols including ethanol are used as fuels and fuel additives in place of at least a portion of petroleum-based products such as gasoline, thereby reducing the need for petroleum. The substitution of alcohols for petroleum-based fuels and fuel additives can conserve natural resources and improve environmental quality, especially when alcohols are produced from feedstocks other than petroleum, such as biomass or natural gas. Ethanol and mixtures of alcohols including ethanol can also be converted into useful chemical industry feedstock olefins, such as ethylene (C₂H₆) and propylene (C₃H₈).

Conversion of syngas to produce organic compounds has been known for many years. Some of the most useful processes are those for direct conversion of syngas to alcohols. Such conversion typically employs heterogeneous catalysts.

U.S. Pat. No. 4,762,858 teaches use of a molybdenum-containing catalyst that consists essentially of molybdenum, ≧one metal selected from among thorium, yttrium, lanthanum, gadolinium, and praseodymium, and optionally ≧one alkali or alkaline earth metal on a support to convert syngas into mixed alcohols containing ethanol and propanol.

In some aspects, this invention is a syngas conversion catalyst that comprises:
a. nickel;
b. two or more metals selected from a group consisting of ruthenium, palladium, gold, chromium, aluminum and tin;
c. a promoter comprising ≧one of an alkali metal or alkaline earth metal; and
d. ≧a catalyst support selected from a group consisting of silica, alumina and magnesium oxide.

“C₂—C₄ alcohols” means one or more alcohols selected from ethanol, propanol, and butanol, including all known isomers of such compounds.

Each syngas conversion catalyst metal may be present in free or combined form. “In free or combined form” means that a metal may be present as a free (or base) metal, an alloy, a compound, an adduct or a combination thereof. Representative compounds include hydroxides, oxides, sulfates, halides, carbides, cyanides, nitrides, nitrates, phosphates, borides, silicides, silicates, oxyhalides, carboxylates (e.g., acetates and acetylacetates), oxalates, carbonates, carbonyls, hydrides, metal-bridged and cluster compounds, and compounds where the metal is part of an anionic or cationic species. Adducts are chemical addition products of two or more distinct molecules.

For any metal, report content as a weight percent (wt %) calculated by taking a ratio of the mass of free metal content of the metal, as a numerator, and mass of all catalyst components as a denominator. Nickel and ≧two metals selected from the group consisting of ruthenium, palladium, gold, chromium, aluminum and tin together are each generally present at lower limits of 0.1 wt %, more preferably 0.25 wt %, most preferably 0.5 wt % and especially 1 wt %. Upper catalyst metal limits are 50 wt %, more preferably 25 wt %, most preferably 10 wt % and especially 8 wt %, each wt % being based upon total free metal content of the catalyst.

Divide number of moles of two or more metals selected from the group consisting of ruthenium, palladium, gold, chromium, aluminum and tin or mixtures by number of moles of nickel to provide a molar ratio thereof. Preferred molar ratios range from a lower level of 1 to 200, more preferably 1 to 1, to an upper level of 8 to 1, more preferably 4 to 1.

Alkali metals include lithium, sodium, potassium, rubidium and cesium. Alkaline earth metals include beryllium, magnesium, calcium, strontium and barium. Cesium represents a preferred promoter, either alone or in combination with calcium.

Divide number of moles of nickel and two or more metals selected from the group consisting of ruthenium, palladium, gold, chromium, aluminum and tin or mixtures by number of moles of alkali and alkaline earth metals to provide a molar ratio thereof. Preferred molar ratios range from a lower level of 1 to 10, more preferably 1 to 3, to an upper level of 10 to 1, more preferably 5 to 1.

The promoter may be present as a metal, oxide, hydroxide, nitride, carbide or as a salt or a combination thereof. The promoter can be incorporated during synthesis gas conversion catalyst preparation by any of a wide variety of ways, such as incipient wetness, dip coating or co-precipitation.

Suitable catalyst supports include silica, alpha-alumina, magnesium oxide, carbon, chromium oxide, titanium oxide, zirconium oxide, and zinc oxide. The catalyst support is present in an amount that is preferably ≧80 wt %, more preferably ≧90 wt % up to (≦) 99 wt %, more preferably ≦98wt %, each wt % being based upon total mass of all catalyst components.

The synthesis gas conversion catalyst can be prepared by a variety of methods known in the art that result in intimate contact among such components, such as incipient wetness (see generally ROBERT L. AUGUSTINE, HETEROGENEOUS CATALYSIS FOR THE SYNTHETIC CHEMIST 184-88 (Marcel Dekker 1996). With incipient wetness, choose sources for catalyst metals to be dispersed on the support from a variety of art-recognized water-soluble or solvent-soluble salts of the metals. Dissolve soluble salt(s) in a quantity of solvent (aqueous, non-aqueous or a combination thereof) to provide a solution; and add sufficient solution to wet, but do no more than fully saturate, the support. Evaporate solvent by applying heat, optionally under vacuum, to leave salt dispersed on the support. Repeat as necessary.

In one embodiment, prepare the synthesis gas conversion catalyst by reducing an initially prepared catalyst precursor composition (formed by combining the nickel; ≧two metals selected from the group consisting of ruthenium, palladium, gold, chromium, aluminum and tin; promoter; and support using the incipient wetness technique) in a reducing atmosphere by flowing a reducing agent such as hydrogen between ambient pressure to moderately elevated pressure (e.g., from 14.7 pounds per square inch gauge (psig) (0.10 megapascals (MPa)) to 600 psig (4.14 MPa)). Such hydrogen treatment has a lower temperature limit of 250 degrees Celsius (° C.), more preferably 330° C. The hydrogen treatment has an upper limit of 1200° C., more preferably 700° C. Repeat the wetting, evaporating and heating steps as needed to achieve a desired concentration of catalytic metal species or promoter on the support.

Use the syngas conversion catalyst in a fixed bed, moving bed, fluidized bed, ebullated bed or a graded bed wherein catalyst concentration or activity varies from inlet to outlet in similar manner to known catalysts. Use the catalyst either as a powder or as a shaped form.

The products of the reaction of CO and H₂ catalyzed by the syngas conversion catalyst include a mixture of C₂—C₄ alcohols, optionally in conjunction with higher alcohols; other products may include methanol, oxygenated organic compounds (oxygenates), hydrocarbons and CO₂. Report product selectivity relative to CO in mole percent (mole %) by carbon atom. For example, for a reaction that converts one mole of CO to 0.2 moles methanol, 0.1 moles ethanol, 0.067 moles n-propanol, 0.2 moles methane, and 0.2 moles carbon dioxide (CO₂) (and 0.233 moles of other products), report the selectivity as 20 mole % for each of these five reaction products.

Selectivity to C₂—C₄ alcohols is preferably higher than selectivity to methanol. In one embodiment, selectivity to methanol is less than one-half selectivity to C₂—C₄ alcohols. In a second embodiment, selectivity to methanol is less than one-fourth selectivity to the C₂—C₄ alcohols. Preferably only small portions of other oxygenates besides alcohols, such as ethers, carboxylic acids, esters, ketones, aldehydes, and peroxides, are formed during syngas conversion. Aldehydes may be hydrogenated to alcohols. In one variation, acetaldehyde can be hydrogenated to ethanol. In another variation, the selectivity to the desired C₂—C₄ alcohols product is 20 percent or above.

Obtaining these selectivity values is generally a matter of varying process conditions and catalyst composition. For example, to increase conversion within the preferred ranges using a reduced catalyst prepared as described above, one may vary one or more of temperature, pressure, gas hourly space velocity (GHSV) and syngas composition to produce a desired result. As conversion increases, product distribution of mixed alcohols produced usually shifts toward higher molecular weight alcohols. Varying recycle ratio and monitoring recycled component content may also alter selectivity. For example, to obtain more C₂—C₄ alcohols in relation to methanol, methanol may be recycled or added to the syngas feed. Varying the catalyst metals themselves may provide the desired selectivity. For example, catalysts comprised of Ni—Ru—Al—Ca—Cs and Ni—Ru—Cr—Ca—Cs on silica produce 3 mole % and 9 mole % ethanol selectivity (based on carbon), respectively, under the same operating conditions wherein the syngas consists of 95 mole percent or above elemental hydrogen and carbon monoxide gases at an H₂/CO molar ratio of 1.0, GHSV corrected to standard temperature and pressure (STP) of 4500 hour⁻¹, temperature of 320° C., and pressure of 500 psig (3.4 MPa), with no recycle.

Generally, selectivity to alcohols depends on pressure. In normal operating ranges, the higher the pressure at a given temperature, the more selective the process will be to mixed alcohols. Operating pressures include pressures of 150 psig (1.03 MPa)) or greater, with pressures in excess of 500 psig (3.44 MPa) being preferred and pressures in excess of 750 psig (5.17 MPa) being more preferred. An especially preferred pressure lies within a range of from 1,500 psig (10.3 MPa) to 4,000 psig (27.6 MPa). Pressures in excess of 4,000 psig (27.6 MPa), while possible, tend to be economically unattractive due to the cost of high pressure vessels, compressors, and energy costs. With that in mind, pressures as high as 20,000 psig (137.9 MPa) are feasible, but a pressure of 10,000 psig (68.9 MPa) or less is preferred and a pressure of 5,000 psig (34.5 MPa) is still more preferred and a pressure of 2,000 psig (13.8 MPa) to 3,000 psig (20.7 MPa) provides very satisfactory results.

Temperatures used in converting syngas to mixed alcohols preferably range from a minimum of 200° C. to a maximum of 500° C. The maximum temperature is more preferably 400° C., and still more preferably 370° C. An especially preferred range of operation is from 240° C. to 350° C.

The GHSV of the syngas feed is a measure of the volume of H₂ plus CO gas at STP passing a given volume of catalyst in one hour. The GHSV is sufficient to produce mixed alcohols and may vary over a very wide range, preferably from 50 hour⁻¹ to 20,000 hour⁻¹. The GHSV is more preferably ≧2000 hour⁻¹, and still more preferably ≧3000 hour⁻¹, but less than or equal to (≦) 10,000 hour⁻¹, more preferably ≦7,500 hour⁻¹. Within the preferred ranges, conversion of syngas usually decreases as GHSV increases. Concurrently, however, productivity usually increases. Measure productivity by mass of product produced per unit volume of catalyst.

At least a portion of unconverted H₂ and CO in effluent gas from the reaction may be recycled to a reactor. Express recycle amount as a ratio of moles of gas in the recycle stream to the moles of gas in a fresh feed stream. Recycle ratios may vary from zero to any number which results in formation of a mixed alcohol product. A recycle ratio of zero is within the scope of the invention with at least some recycle preferred. After separation of the desired alcohols, if at least a portion of the effluent gas is recycled and it contains unconverted H₂ and CO, it is preferable to remove water, CO₂, and even more preferably any hydrocarbons formed. The recycle of methanol may favor production of C₂—C₄ mixed alcohols. In another variation, one or more C₂—C₄ alcohols or other alcohols may be recycled to form higher alcohols.

### EXAMPLE 1

Combine 5.6 milligrams (mg) nickel(II) nitrate hexahydrate (Ni(NO₃)₂·6H₂O), 8.8 mg palladium(II) nitrate dihydrate (Pd(NO₃)₂·2H₂O), 7.0 mg aluminum nitrate nonahydrate (Al(NO₃)₃·9H₂O), and 250 mg of silica gel (SiO₂) with a particle size between 60 mesh (0.250 millimeter (mm) sieve opening) and 100 mesh (0.149 mm sieve opening) into 0.31 milliliters (mL) of water. Heat the mixture at 70° C. for 2 hours under vacuum, allowing the water to evaporate to dryness. Dissolve 12.4 mg cesium nitrate (CsNO₃) in 0.310 mL of water, and combine this second mixture with the first mixture. Heat for 2 hours at 70° C. under vacuum, then increase the heat in static air with a 10° C. per minute heating rate until the temperatures reaches 120° C. and hold for 2 hours. Finally, heat to 350° C. with a 10° C. per minute heating rate and hold for 2 hours. Cool the finished catalyst to ambient temperature.

Load 0.2 mL of this catalyst into a ¼-inch (0.635 cm) stainless steel tubular reactor. Reduce the catalyst in-situ with flowing H₂ gas at 330 ° C. for 150 minutes. Heat the reactor and its contents to the temperature stated in Table I using an electric furnace. Use premixed H₂ and CO to pressurize the interior of the reactor to the pressure stated in Table I. The feed gas mixture contains H₂ and CO at the ratios stated in Table I. Pass the feed gas mixture at the stated GHSVs through the reactor to yield a reaction product. Pass the reaction product through a pressure letdown valve and flow past a gas chromatograph (GC) sampling point into a cooled condenser. Collect and analyze both gaseous and liquid products from the condenser. GC analysis of reaction products from the reaction chamber shows that MeOH, EtOH and other alcohols are present. Table I shows product content in terms of molar selectivity to ethanol, all alcohols (ROH selectivity), hydrocarbons and CO₂ together with reaction temperature and CO conversion. As used herein, “MeOH/ROH” means the fraction of alcohols that is attributed to methanol, on a molar basis by carbon atom.

TABLE 1

| | |
|---|---|
| Temperature (° C.) | 320 |
| Pressure (psig) | 500 |
| H2/CO (molar ratio) | 1 |
| GHSV (hour⁻¹) | 4500 |
| CO Conversion (wt %) | 1.3 |
| Ethanol selectivity (mole %) | 18 |
| ROH selectivity (mole %) | 45 |
| MeOH/ROH (molar ratio) | 0.6 |
| Acetaldehyde selectivity (mole %) | 33 |
| Selectivity to hydrocarbons (mole %) | 22 |
| Selectivity to CO₂ (mole %) | 0 |

### EXAMPLE 2

Replicate Example 1, but feed 15.2 mg ruthenium(III) acetylacetonate (Ru(C₅H₇O₂)₃) rather than Pd(NO₃)₂·2H₂O, and feed 7.7 mg chromium(III) nitrate nonahydrate (Cr(NO₃)₃·9H₂O) rather than Al(NO₃)₃·9H₂O. The promoter is a combination of 5.0 mg calcium nitrate tetrahydrate (Ca(NO₃)₃·4H₂O) and 12.6 mg CsNO₃ rather than CsNO₃ alone.

The experimental procedure for making alcohols is the same as Example 1. The results are reported in Table 2.

TABLE 2

| | | | |
|---|---|---|---|
| Temperature (° C.) | 300 | 320 | 340 |
| Pressure (psig) | 500 | 500 | 500 |
| H2/CO (molar ratio) | 1 | 1 | 1 |
| GHSV (hour⁻¹) | 4500 | 4500 | 4500 |
| CO Conversion (wt %) | 2 | 2.7 | 2.1 |
| Ethanol selectivity (mole %) | 9 | 9 | 15 |
| ROH selectivity (mole %) | 16 | 15 | 20 |
| MeOH/ROH (molar ratio) | 0.3 | 0.27 | 0.22 |
| Acetaldehyde selectivity (mole %) | 4 | 4 | 6 |
| Selectivity to hydrocarbons (mole %) | 54 | 54 | 63 |
| Selectivity to CO₂ (mole %) | 24 | 25 | 9 |

The data in Examples 1 and 2 show syngas conversion to alcohols even at relatively low pressures such as 500 psig (3.4 MPa) or less.

1. A synthesis gas conversion catalyst comprising:
nickel;
two or more metals selected from a group consisting of ruthenium, palladium, gold, chromium, aluminum and tin;
a promoter comprising at least one of an alkali metal or alkaline earth metal; and
a catalyst support selected from a group consisting of silica, alumina, and magnesium oxide, or a mixture thereof.

2. The catalyst of claim 1, wherein the two or more metals comprise palladium and aluminum; or ruthenium and chromium; or gold and aluminum.

3. The catalyst of claim 1, wherein the promoter is cesium.

4. The catalyst of claim 3, wherein the promoter further comprises calcium.

5. A process for producing one or more C₂—C₄ alcohols, which method comprises placing synthesis gas in contact with the catalyst of claim 1 under conditions sufficient to convert at least a portion of the synthesis gas to at least one of ethanol, propanol and butanol.

6. The process for producing C₂—C₄ alcohols according to claim 5, wherein the catalyst is reduced using a reducing agent prior to contact with the synthesis gas.

7. The process for producing C₂—C₄ alcohols according to claim 5, wherein at least a portion of the synthesis gas is converted to methanol.

8. The process for producing C₂—C₄ alcohols according to claim 5, wherein at least a portion of the synthesis gas is converted to acetaldehyde.

9. The process for producing C₂—C₄ alcohols according to claim 5, wherein the conditions include a minimum pressure of 500 psig (3.4 MPa).
